(19) United States
(12) Patent Application Publication
(10) Pub. No.: US 2017/0202874 A1
(43) Pub. Date: Jul. 20, 2017

(54) POLYMERS FOR THERMALLY-REGULATED RELEASE OF HNO AND OTHER MOLECULES

(71) Applicants: University of Washington, Seattle, WA (US); Pacific Lutheran University, Tacoma, WA (US)

(72) Inventors: Andrew J. Boydston, Seattle, WA (US); Neal A. Yakelis, Federal Way, WA (US); Derek C. Church, Seattle, WA (US)

(73) Assignees: University of Washington, Seattle, WA (US); Pacific Lutheran University, Tacoma, WA (US)

(21) Appl. No.: 15/405,988

(22) Filed: Jan. 13, 2017

Related U.S. Application Data
(60) Provisional application No. 62/279,216, filed on Jan. 15, 2016.

(51) Int. Cl. A61K 33/00; A61N 5/06; A61K 41/00

(52) U.S. Cl. CPC A61K 33/00; A61K 47/48176; A61K 41/0028; A61N 5/062; A61N 2005/0661

(57) ABSTRACT

Multivalent scaffolds configured to facilitate drug release upon exposure to a stimulus, such as heat, or light, are described herein. The multivalent scaffolds are covalently bound to a moiety that is susceptible to decomposition upon exposure to stimulus. The moiety releases HNO upon decomposition. In some embodiments, the moiety is in turn linked to an agent to be delivered, such as a therapeutic agent, which is released from the multivalent scaffolds when the moiety decomposes. In some embodiments, the moiety is a 1,2-oxazine moiety. In some embodiments, the multivalent scaffold is a polymer. A plurality of 1,2-oxazine moieties can be covalently bound as side chains to the backbone of the polymer.

### CROSS-REFERENCE TO RELATED APPLICATION

This application claims the benefit of U.S. Patent Application No. 62/279,216, filed Jan. 15, 2016, the disclosure of which is hereby incorporated by reference in its entirety.

### STATEMENT OF GOVERNMENT LICENSE RIGHTS

This invention was made with U.S. government support under DMR-1452726, awarded by the National Science Foundation. The U.S. Government has certain rights in the invention.

### BACKGROUND

Polymer-based drug delivery systems are an area of continuous research as these systems can be engineered to enable advanced capabilities, including site-specific cellular targeting, stabilization of small molecule cargo, systemic transport throughout the body, and controlled kinetic profiles for small molecule release. In many cases, the use of either exogenous or endogenous stimuli has been demonstrated for achieving spatiotemporal control over drug release from polymer-based nanomedicines. Thermal activation, for example, is particularly attractive considering that the technology for localized hyperthermia can be found in the forms of nanoparticle-based photo- or magneto-thermal transduction, photothermal organic dyes and through application of high intensity focused ultrasound. Although many examples of thermally-induced drug delivery from polymer-based systems have been demonstrated in the literature, most have involved phase transitions taking advantage of a lower critical solution temperature (LCST) polymer to rapidly expel encapsulated small molecules. Importantly, these designs generally do not allow for covalent drug attachment, which can compromise the fidelity of the delivery system and give rise to unpredictable release kinetics. Additionally, LCSTs are highly dependent upon environmental conditions, such as salt concentration and pH.

There is a need for a stable carrier that can provide predictable and regulated drug release upon exposure of a given stimuli. This present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, this disclosure features a HNO-releasing compound, including a multivalent scaffold; and a plurality of 1,2-oxazine pendant moieties independently covalently bound to the scaffold, the 1,2-oxazine pendant moieties having any one of Formula (I-A), (I-B), (I-C), (I-D), (I-E), or (I-F),

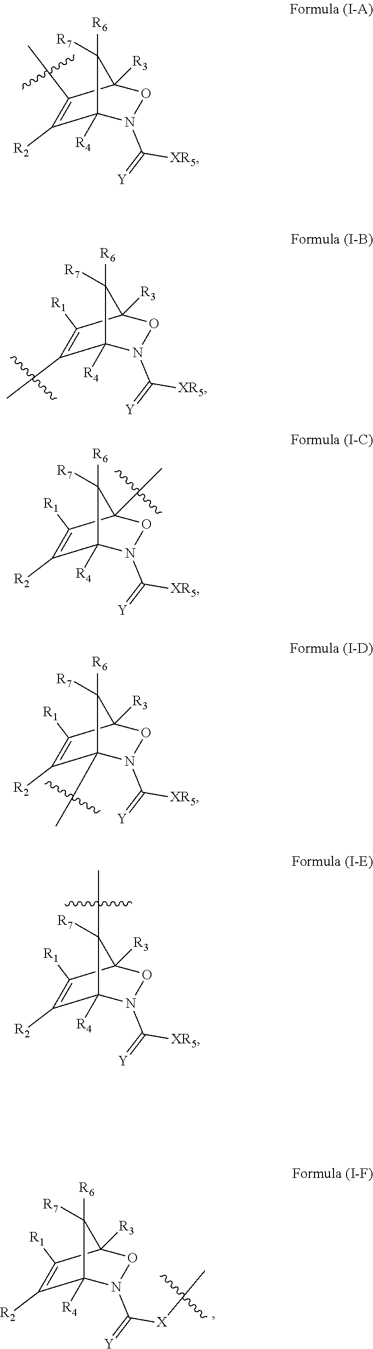

wherein

R₁, when present, is independently selected from a fluorescent molecule, a signaling agent, and a therapeutic agent, each optionally covalently bonded to a self-immolative linker, or R₁ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₂, when present, is independently selected from a fluorescent molecule, a signaling agent, and a therapeutic agent, each optionally covalently bonded to a self-immolative linker, or R₂ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₃, when present, is independently selected from a fluorescent molecule, a signaling agent, and a therapeutic agent, each optionally covalently bonded to a self-immolative linker, or R₃ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₄, when present, is independently selected from a fluorescent molecule, a signaling agent, and a therapeutic agent, each optionally covalently bonded to a self-immolative linker, or R₄ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₆, when present, is independently selected from a fluorescent molecule, a signaling agent, and a therapeutic agent, each optionally covalently bonded to a self-immolative linker, or R₆ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₇, when present, is independently selected from a fluorescent molecule, a signaling agent, and a therapeutic agent, each optionally covalently bonded to a self-immolative linker, or R₇ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₅, when present, is selected from a fluorescent molecule, a signaling agent, and a therapeutic agent, each optionally covalently bonded to a self-immolative linker; or

R₅, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

Y is selected from O, NH, NC₁₋₆alkyl, and S; and

X is selected from O and NH;

wherein each 1,2-oxazine pendant moiety is configured to release one HNO molecule upon decomposition.

In some embodiments, at least one of R₁, R₂, R₃, R₄, R₅, R₆, or R₇, when present, is selected from a fluorescent molecule, a signaling agent, and a therapeutic agent, each optionally covalently bonded to a self-immolative linker, and the 1,2-oxazine moiety is configured to release the fluorescent molecule, the signaling agent, or the therapeutic agent.

In another aspect, the present disclosure features a composition, including a triggering agent selected from a photothermal dye and a nanoparticle; and the HNO-releasing compound above.

In yet another aspect, the present disclosure features a method of releasing HNO, including: exposing a compound of claim 1 to a triggering event, decomposing a compound described above, and releasing HNO from the compound.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a scheme illustrating a route for thermal decomposition of 1,2-oxazine Diels-Alder adducts.

FIG. 2A is a scheme illustrating the use of 1,2-oxazines as a tool for polymer conjugation (top) and as a trigger for SIP depolymerization (bottom).

FIG. 2B is a scheme illustrating a generalized design for polymer scaffold capable of releasing multiple units of HNO and small molecule.

FIG. 3 is a scheme illustrating a synthesis for a copolymer of the present disclosure.

FIG. 4A is a graph showing UV-vis spectra from the thermolysis of an embodiment of a polymer of the present disclosure in pH 7.5 phosphate buffered H₂O at 60° C. (Topmost curve at ˜329 nm wavelength is at time=0 h, topmost curve at ˜390 nm wavelength is at 945 h (39 d)).

FIG. 4B is a graph showing release of p-nitroaniline from an embodiment of the present disclosure in pH 7.5 phosphate buffered H₂O. Error bars represent first standard deviation (average of three runs). Legend: black solid circles=60° C., white squares=37° C., white diamonds=22° C., black solid squares=4° C.

### DETAILED DESCRIPTION

Multivalent scaffolds configured to facilitate drug release upon exposure to a stimulus, such as heat, or light, are described herein. The multivalent scaffolds are covalently bound to a moiety that is susceptible to decomposition upon exposure to stimulus. The moiety releases HNO upon decomposition. In some embodiments, the moiety is in turn linked to an agent to be delivered, such as a therapeutic agent, which is released from the multivalent scaffolds when the moiety decomposes. In some embodiments, the moiety is a 1,2-oxazine moiety. In some embodiments, the multivalent scaffold is a polymer. A plurality of 1,2-oxazine moieties can be covalently bound as side chains to the backbone of the polymer.

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

“Optionally substituted” groups can refer to, for example, functional groups that may be substituted or unsubstituted by additional functional groups. For example, when a group is unsubstituted, it can be referred to as the group name, for example alkyl or aryl. When a group is substituted with additional functional groups, it may more generically be referred to as substituted alkyl or substituted aryl.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(O)C₁-C₆ alkyl indicate the same functionality; similarly arylalkyl and alkylaryl indicate the same functionality.

As used herein, the term “alkyl” refers to a straight or branched hydrocarbon groups. In some embodiments, an alkyl group contains from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms. In some embodiments, alkyl has 1 to 10 carbon atoms (e.g., 1 to 8 carbon atoms, 1 to 6 carbon atoms, 1 to 3 carbon atoms, 1 or 2 carbon atoms, or 1 carbon atom). Representative alkyl groups include methyl, ethyl, propyl (e.g., n-propyl, isopropyl), butyl (e.g., n-butyl, sec-butyl, and tert-butyl), pentyl (e.g., n-pentyl, tert-pentyl, neopentyl, isopentyl, pentan-2-yl, pentan-3-yl), and hexyl (e.g., n-hexyl and isomers) groups.

As used herein, the term “alkylene” refers to a linking alkyl group (e.g., a divalent linking alkyl group).

As used herein, the term “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cy cl op entenyl, cyclohexenyl, cyclohexadienyl, cycloheptatrienyl, norbomyl, norpinyl, norcamyl, adamantyl, and the like.

As used herein, the term “cycloalkylene” refers to a linking cycloalkyl group.

As used herein, the term “perfluoroalkyl” refers to straight or branched fluorocarbon chains. In some embodiments, perfluoroalkyl has 1 to 10 carbon atoms (e.g., 1 to 8 carbon atoms, 1 to 6 carbon atoms, 1 to 3 carbon atoms, 1 or 2 carbon atoms, or 1 carbon atom). Representative alkyl groups include trifluoromethyl, pentafluoroethyl, etc.

As used herein, the term “perfluoroalkylene” refers to a linking perfluoroalkyl group.

As used herein, the term “heteroalkyl” refers to a straight or branched chain alkyl groups and where one or more of the carbon atoms is replaced with a heteroatom selected from O, N, or S. In some embodiments, heteroalkyl alkyl has 1 to 10 carbon atoms (e.g., 1 to 8 carbon atoms, 1 to 6 carbon atoms, 1 to 3 carbon atoms, 1 or 2 carbon atoms, or 1 carbon atom).

As used herein, the term “heteroalkylene” refers to a linking heteroalkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group (e.g., a divalent linking alkenyl group).

As used herein, the term “alkoxy” refers to an alkyl or cycloalkyl group as described herein bonded to an oxygen atom. In some embodiments, alkoxy has 1 to 10 carbon atoms (e.g., 1 to 8 carbon atoms, 1 to 6 carbon atoms, 1 to 3 carbon atoms, 1 or 2 carbon atoms, or 1 carbon atom). Representative alkoxy groups include methoxy, ethoxy, propoxy, and isopropoxy groups.

As used herein, the term “perfluoroalkoxy” refers to a perfluoroalkyl or cyclic perfluoroalkyl group as described herein bonded to an oxygen atom. In some embodiments, perfluoroalkoxy has 1 to 10 carbon atoms (e.g., 1 to 8 carbon atoms, 1 to 6 carbon atoms, 1 to 3 carbon atoms, 1 or 2 carbon atoms, or 1 carbon atom). Representative perfluoroalkoxy groups include trifluoromethoxy, pentafluoroethoxy, etc.

As used herein, the term “aryl” refers to an aromatic hydrocarbon group having 6 to 10 carbon atoms. Representative aryl groups include phenyl groups. In some embodiments, the term “aryl” includes monocyclic or polycyclic (e.g., having 2, 3, or 4 fused rings) aromatic hydrocarbons such as, for example, phenyl, naphthyl, anthracenyl, phenanthrenyl, indanyl, and indenyl.

As used herein, the term “arylene” refers to a linking aryl group (e.g., a divalent linking aryl group). For example, the term “phenylene” refers to a linking phenyl group.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heteroarylene” refers to a linking heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, an ether linkage refers to a linking —O— group.

As used herein, a carbamate linkage refers to a linking —NC(O)O— group (both forward and backward directions).

As used herein, a carbonate linkage refers to a linking —OC(O)O— group.

As used herein, an amide linkage refers to a linking —NHC(O)— group (both forward and backward directions).

As used herein, an ester linkage refers to a linking —C(O)O— group (both forward and backward directions).

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer while the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Compounds and Polymers

In one aspect, the present disclosure features a HNO-releasing compound, including

(a) a multivalent scaffold; and

(b) a plurality of 1,2-oxazine pendant moieties independently covalently bound to the scaffold, the 1,2-oxazine pendant moieties having any one of Formula (I-A), (I-B), (I-C), (I-D), (I-E), or (I-F),

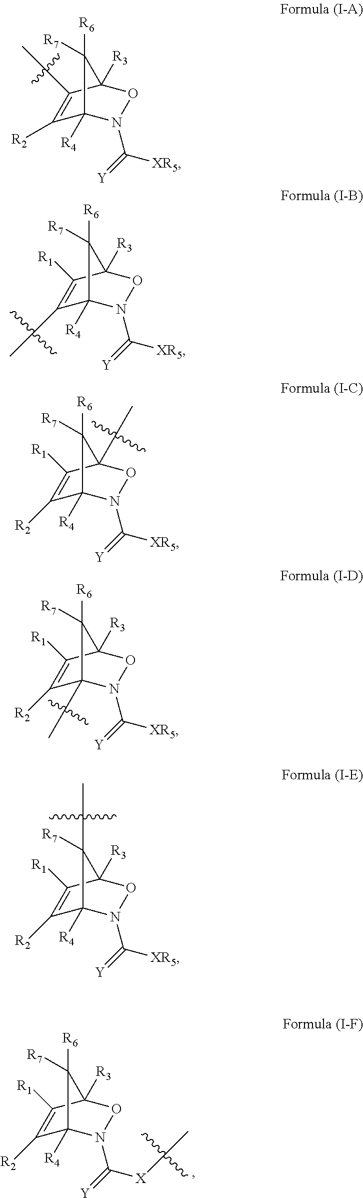

wherein

R₁, when present, is independently selected from a fluorescent molecule, a signaling agent, and a therapeutic agent, each optionally covalently bonded to a self-immolative linker, or R₁ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₂, when present, is independently selected from a fluorescent molecule, a signaling agent, and a therapeutic agent, each optionally covalently bonded to a self-immolative linker, or R₂ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₃, when present, is independently selected from a fluorescent molecule, a signaling agent, and a therapeutic agent, each optionally covalently bonded to a self-immolative linker, or R₃ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₄, when present, is independently selected from a fluorescent molecule, a signaling agent, and a therapeutic agent, each optionally covalently bonded to a self-immolative linker, or R₄ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₆, when present, is independently selected from a fluorescent molecule, a signaling agent, and a therapeutic agent, each optionally covalently bonded to a self-immolative linker, or R₆ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₇, when present, is independently selected from a fluorescent molecule, a signaling agent, and a therapeutic agent, each optionally covalently bonded to a self-immolative linker, or R₇ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₅, when present, is selected from a fluorescent molecule, a signaling agent, and a therapeutic agent, each optionally covalently bonded to a self-immolative linker; or

R₅, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

Y is selected from O, NH, NC₁₋₁₀alkyl, and S; and

X is selected from O and NH;

wherein each 1,2-oxazine pendant moiety is configured to release one HNO molecule upon decomposition.

In some embodiments, when the multivalent scaffold is a polymer, the plurality of 1,2-oxazine pendant moieties is covalently bound as side chains off of a polymer backbone, and not as end groups that cap the termini of a given polymer chain. For example, the 1,2-oxazine pendant moieties can be covalently attached to repeating units forming the backbone of the polymer.

As used herein, the point of covalent attachment of the 1,2-oxazine pendant moieties is shown by

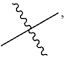

where the straight bond is bound at one end to the 1,2-oxazine moiety, and the other end is bound to the scaffold, optionally via a linker.

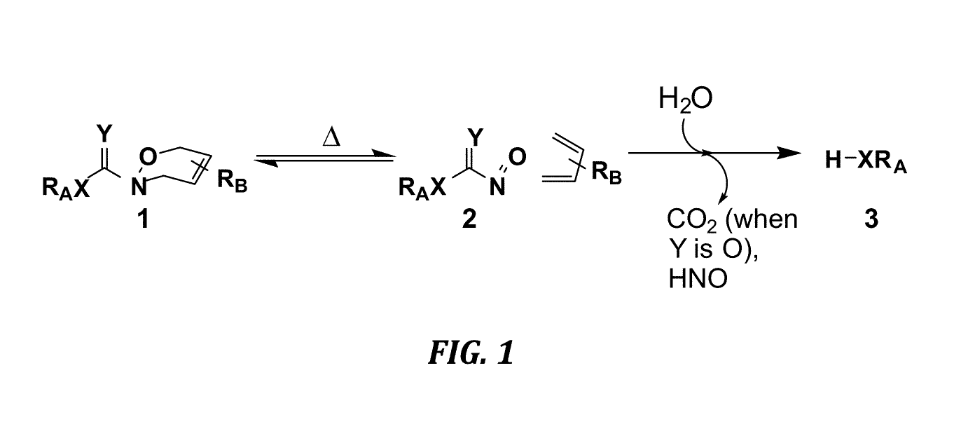

Without wishing to be bound by theory, it is believed that 1,2-oxazine moieties decompose as shown in exemplary FIG. 1. Referring to FIG. 1, in some embodiments, upon exposure to a stimulus, such as heat (Δ), a cycloaddition adduct 1 can undergo cycloreversion to release moiety 2, when is then hydrolytically degraded to release CO₂ (when Y is O), HNO, and H—XR_{A}, where R_{A} is a fluorescent molecule, a signaling molecule, or a therapeutic agent.

When pendant on a multivalent scaffold, the decomposition of 1,2-oxazine moieties is shown in Scheme 1, below.

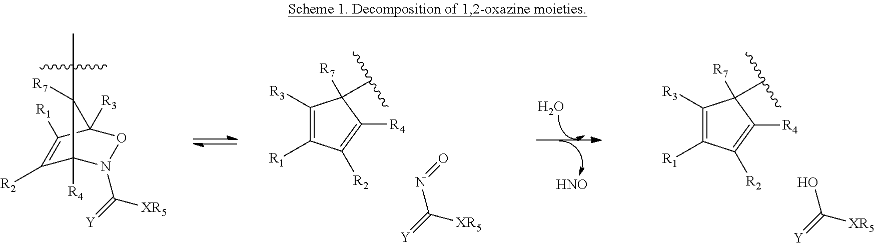

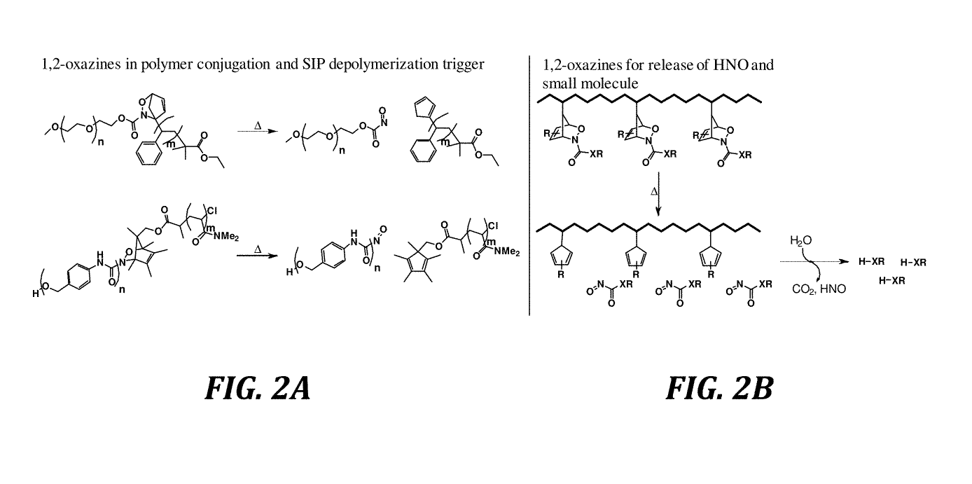

Referring to FIG. 2B, in some embodiments, when the scaffold is a polymer (depicted a polyethylene for illustration purposes), the covalently bound 1,2-oxazine moieties degrade through thermal stimulus and release a molecule (e.g., RXC(O)N═O) that ultimately degrades into HNO, CO₂, and a therapeutic and/or signaling molecule.

In some embodiments, the multivalent scaffold is selected from a polymer, a nanoparticle, a micelle, a liposome, a microbubble, a hydrogel, and electrospun fiber. In one embodiment, the multivalent scaffold is a polymer. In certain embodiments, the present disclosure is directed to a polymer including a polymer backbone; and a plurality of pendant 1,2-oxazine moieties covalently coupled to the polymer backbone.

In some embodiments, the polymer to which the pendant 1,2-oxazine moieties are covalently bound is selected from a poly(olefin), a poly(cyclic olefin), a poly(ester), a poly(ether), a poly(urethane), a poly(acrylate), a poly(acrylamide), and a poly(C₁₋₃ alkyl acrylate). In certain embodiments, the polymer is a poly(cyclic olefin). For example, the poly(cyclic olefin) can be a poly(norbornene) of Formula (II)

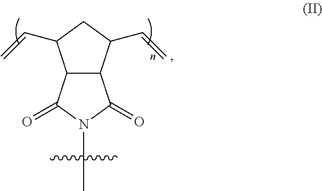

wherein n is an integer from 5-20,000.

In some embodiments, n is an integer from 5 to 10,000 (e.g., from 5 to 5,000, from 5 to 1,000, from 5 to 500, from 5 to 100, or from 5 to 50).

In some embodiments, the polymer to which the pendant 1,2-oxazine moieties are covalently bound is selected from polynorbornene, polymethacrylate, polymethylmethacrylate, polyester, and vinyl polymers. In certain embodiments, the polymer, such as poly(norbornene), is assembled through ring-opening metathesis.

In some embodiments, the 1,2-oxazine pendant moieties independently covalently bound to the scaffold have Formula (I-A).

In some embodiments, the 1,2-oxazine pendant moieties independently covalently bound to the scaffold have Formula (I-B).

In some embodiments, the 1,2-oxazine pendant moieties independently covalently bound to the scaffold have Formula (I-C).

In some embodiments, the 1,2-oxazine pendant moieties independently covalently bound to the scaffold have Formula (I-D).

In some embodiments, the 1,2-oxazine pendant moieties independently covalently bound to the scaffold have Formula (I-E).

In some embodiments, the 1,2-oxazine pendant moieties independently covalently bound to the scaffold have Formula (I-F).

In some embodiments, at least one of R₁, R₂, R₃, R₄, R₅, R₆, or R₇, when present, is selected from a fluorescent molecule, a signaling agent, and a therapeutic agent, each optionally covalently bonded to a self-immolative linker.

In some embodiments, at least one of R₁, R₂, R₃, R₄, R₅, R₆, or R₇, when present, is selected from a signaling agent and a therapeutic agent, each optionally covalently bonded to a self-immolative linker.

In some embodiments, at least one of R₁, R₂, R₃, R₄, R₅, R₆, or R₇, when present, is a therapeutic agent optionally covalently bonded to a self-immolative linker.

In some embodiments, at least one of R₁, R₂, R₃, R₄, R₅, R₆, or R₇, when present, is selected from a fluorescent molecule, a signaling agent, and a therapeutic agent.

In some embodiments, at least one of R₁, R₂, R₃, R₄, R₅, R₆, or R₇, when present, is selected from a signaling agent and a therapeutic agent.

In some embodiments, at least one of R₁, R₂, R₃, R₄, R₅, R₆, or R₇, when present, is a therapeutic agent.

In some embodiments, one of R₁, R₂, R₃, R₄, R₅, R₆, or R₇, when present, is selected from a fluorescent molecule, a signaling agent, and a therapeutic agent, each optionally covalently bonded to a self-immolative linker.

In some embodiments, one of R₁, R₂, R₃, R₄, R₅, R₆, or R₇, when present, is selected from a signaling agent and a therapeutic agent, each optionally covalently bonded to a self-immolative linker.

In some embodiments, one of R₁, R₂, R₃, R₄, R₅, R₆, or R₇, when present, is a therapeutic agent optionally covalently bonded to a self-immolative linker.

In some embodiments, one of R₁, R₂, R₃, R₄, R₅, R₆, or R₇, when present, is selected from a fluorescent molecule, a signaling agent, and a therapeutic agent.

In some embodiments, one of R₁, R₂, R₃, R₄, R₅, R₆, or R₇, when present, is selected from a signaling agent and a therapeutic agent.

In some embodiments, one of R₁, R₂, R₃, R₄, R₅, R₆, or R₇, when present, is a therapeutic agent.

In some embodiments, the 1,2-oxazine pendant moieties having any one of Formula (I-A), (I-B), (I-C), (I-D), or (I-E), and

R₁, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₂, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₃, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₄, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₆, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₇, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl; and

R₅ is selected from a fluorescent molecule, a signaling agent, and a therapeutic agent, wherein the fluorescence molecule, the signaling agent, or the therapeutic agent is optionally covalently bonded to a self-immolative linker.

In some embodiments, the 1,2-oxazine pendant moieties having any one of Formula (I-A), (I-B), (I-C), (I-D), or (I-E), and

R₁, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₂, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₃, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₄, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₆, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

R₇, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl; and

R₅ is selected from a fluorescent molecule, a signaling agent, and a therapeutic agent, wherein the fluorescence molecule, the signaling agent, or the therapeutic agent is optionally covalently bonded to a self-immolative linker.

In some embodiments, R₁, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl.

In some embodiments, R₁, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₁, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₁, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₁, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl.

In some embodiments, R₁, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₁, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₁, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₁, when present, is independently selected from H and C₁₋₆ alkyl, wherein the C₁₋₆ alkyl is optionally substituted with 1, 2, 3, or 4 halo.

In some embodiments, R₁, when present, is independently selected from H and methyl.

In some embodiments, R₁, when present, is H.

In some embodiments, R₂, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl.

In some embodiments, R₂, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₂, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₂, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₂, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl.

In some embodiments, R₁, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₂, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₂, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₂, when present, is independently selected from H and C₁₋₆ alkyl, wherein the C₁₋₆ alkyl is optionally substituted with 1, 2, 3, or 4 halo.

In some embodiments, R₂, when present, is independently selected from H and methyl.

In some embodiments, R₂, when present, is H.

In some embodiments, R₃, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl.

In some embodiments, R₃, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₃, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₃, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₃, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl.

In some embodiments, R₃, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₃, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₃, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₃, when present, is independently selected from H and C₁₋₆ alkyl, wherein the C₁₋₆ alkyl is optionally substituted with 1, 2, 3, or 4 halo.

In some embodiments, R₃, when present, is independently selected from H and methyl.

In some embodiments, R₃, when present, is H.

In some embodiments, R₄, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl.

In some embodiments, R₄, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₄, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₄, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₄, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl. In some embodiments, R₄, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₄, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₄, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₄, when present, is independently selected from H and C₁₋₆ alkyl, wherein the C₁₋₆ alkyl is optionally substituted with 1, 2, 3, or 4 halo.

In some embodiments, R₄, when present, is independently selected from H and methyl.

In some embodiments, R₄, when present, is H.

In some embodiments, R₆, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl.

In some embodiments, R₆, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₆, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₆, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₆, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl.

In some embodiments, R₆, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₆, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₆, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₆, when present, is independently selected from H and C₁₋₆ alkyl, wherein the C₁₋₆ alkyl is optionally substituted with 1, 2, 3, or 4 halo.

In some embodiments, R₆, when present, is independently selected from H and methyl.

In some embodiments, R₆, when present, is H.

In some embodiments, R₇, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl.

In some embodiments, R₇, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₇, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₇, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₇, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl.

In some embodiments, R₇, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₇, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₇, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₇, when present, is independently selected from H and C₁₋₆ alkyl, wherein the C₁₋₆ alkyl is optionally substituted with 1, 2, 3, or 4 halo.

In some embodiments, R₇, when present, is independently selected from H and methyl.

In some embodiments, R₇, when present, is H.

In some embodiments, Y is selected from O, NH, and NC₁₋₆alkyl.

In some embodiments, Y is selected from O and NH.

In some embodiments, Y is O.

In some embodiments, Y is NH.

In some embodiments, X is O.

In some embodiments X is NH.

In some embodiments, R₅, when present, is selected from a fluorescent molecule, a signaling agent, and a therapeutic agent, each optionally covalently bonded to a self-immolative linker. In some embodiments, R₅, when present, is selected from a fluorescent molecule, a signaling agent, and a therapeutic agent. In some embodiments, R₅, when present, is selected from a fluorescent molecule and a therapeutic agent. In some embodiments, R₅, when present, is a therapeutic agent. As used herein, a self-immolative linker refers to a linker, which can include an oligomeric or polymeric linking segment that decomposes in a controlled manner. In some embodiments, the self-immolative linker has a head end, a tail end, and a plurality of repeating units, and the repeating units are configured to decompose sequentially in a head-to-tail direction. Examples of self-immolative polymers are described, for example, in PCT application No. PCT/US2013/073928, filed Dec. 9, 2013, herein incorporated in its entirety.

In some embodiments, R₅, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl

In some embodiments, R₅, when present, is independently selected from H, C₁₋₆ alkyl, C₂₋₆ alkenyl, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;

In some embodiments, R₅, when present, is independently selected from H, C₁₋₆ alkyl, C₂₋₆ alkenyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₅, when present, is independently selected from H, C₁₋₆ alkyl, and halo, wherein the C₁₋₆ alkyl, or C₂₋₆ alkenyl is each optionally substituted with 1, 2, 3, or 4 substituents each independently selected from halo, hydroxy, OMe, OAc, CN, NO₂, and CO₂C₁₋₆ alkyl.

In some embodiments, R₅, when present, is independently selected from H and C₁₋₆ alkyl, wherein the C₁₋₆ alkyl is optionally substituted with 1, 2, 3, or 4 substituents each independently selected from halo, hydroxy, OMe, OAc, CN, NO₂, and CO₂C₁₋₆ alkyl.

In some embodiments, R₅, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 substituents each independently selected from halo, hydroxy, and OMe.

In some embodiments, R₅, when present, is independently selected from H and C₁₋₆ alkyl, wherein the C₁₋₆ alkyl is optionally substituted with 1, 2, 3, or 4 substituents each independently selected from halo, hydroxy, and OMe.

In some embodiments, R₅, when present, is independently selected from H, C₁₋₆ alkyl, C₂₋₆ alkenyl, wherein the C₁₋₆ alkyl, or C₂₋₆ alkenyl is each optionally substituted with 1, 2, 3, or 4 halo.

In some embodiments, R₅, when present, is independently selected from H and C₁₋₆ alkyl, wherein the C₁₋₆ alkyl is optionally substituted with 1, 2, 3, or 4 halo.

In some embodiments, R₅, when present, is independently selected from H and methyl.

In some embodiments, R₅, when present, is H.

In some embodiments, the 1,2-oxazine moiety has any one of Formula (I-A), (I-B), (I-C), (I-D), or (I-E), and R₅ is selected from a fluorescent molecule, a signaling agent, and a therapeutic agent, each optionally covalently bonded to a self-immolative linker. In some embodiments, the 1,2-oxazine moiety has any one of Formula (I-A), (I-B), (I-C), (I-D), or (I-E), and R₅ is selected from a fluorescent molecule, a signaling agent, and a therapeutic agent. In some embodiments, the 1,2-oxazine moiety has any one of Formula (I-A), (I-B), (I-C), (I-D), or (I-E), and R₅ is selected from a fluorescent molecule and a therapeutic agent. In some embodiments, the 1,2-oxazine moiety has any one of Formula (I-A), (I-B), (I-C), (I-D), or (I-E), and R₅ is a therapeutic agent.

In some embodiments, the 1,2-oxazine moiety is configured to release the fluorescent molecule, the signaling agent, or the therapeutic agent. The therapeutic agent can be selected from an analgesic agent, an anticancer therapeutic agent, and a non-steroidal anti-inflammatory drug (NSAID). For example, the therapeutic agent can be selected from salicylate NSAID, propionic acid NSAID, acetic acid NSAID derivatives, anthranilic acid NSAID. In some embodiments, the therapeutic agent is selected from p-nitroaniline, ibuprofen, (S)-naproxen, indomethacin, meclofenamic acid, doxorubicin and celecoxib.

In some embodiments, the 1,2-oxazine moiety is covalently bonded to the multivalent scaffold via a first linker L₁. The first linker L₁ can be selected from C₁₋₂₀ alkylene, C₂₋₂₀ alkenylene, C₂₋₂₀ alkynylene, an ether linkage, a carbamate linkage, a carbonate linkage, an amide linkage and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene. In some embodiments, the first linker L₁ is selected from C₁₋₂₀ alkylene, C₂₋₂₀ alkenylene, C₂₋₂₀ alkynylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, C₂₋₂₀ alkynylene, or a divalent poly(C₁₋₄ alkylene glycol) linkage. In some embodiments, the first linker L₁ is selected from C₁₋₂₀ alkylene, C₂₋₂₀ alkenylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or a divalent poly(C₁₋₄ alkylene glycol) linkage. In some embodiments, the first linker L₁ is selected from C₁₋₂₀ alkylene, a divalent poly(C₁₋₄ alkylene glycol) linkage, an amide linkage, and an ester linkage, wherein said amide linkage, or ester linkage is optionally substituted with 1 or 2 C₁₋₂₀ alkylene or a divalent poly(C₁₋₄ alkylene glycol) linkage. In some embodiments, the first linker L₁ is selected from C₁₋₂₀ alkylene, an amide linkage, and an ester linkage, wherein said amide linkage, or ester linkage is optionally substituted with 1 or 2 C₁₋₂₀ alkylene. In some embodiments, the first linker L₁ is a C₁₋₂₀ alkylene.

In some embodiments, the multivalent scaffold further includes hydrophilic pendant moieties, such as poly(ethylene glycol) or a C₁₋₃ alkoxy poly(ethylene glycol). The hydrophilic pendant moieties can be present in an amount of from 20 weight percent or more (30 weight percent or more, 40 weight percent or more, 50 weight percent or more, 60 weight percent or more, 70 weight percent or more, or 80 weight percent or more) and/or 90 weight percent or less (80 weight percent or less, 70 weight percent or less, 60 weight percent or less, 50 weight percent or less, 40 weight percent or less, or 30 weight percent or less) relative to the weight of the HNO-releasing compound. In some embodiments, when the multivalent scaffold is a polymer, the repeating units including hydrophilic pendant moieties can be present in an amount of from 20 weight percent or more (30 weight percent or more, 40 weight percent or more, 50 weight percent or more, 60 weight percent or more, 70 weight percent or more, or 80 weight percent or more) and/or 90 weight percent or less (80 weight percent or less, 70 weight percent or less, 60 weight percent or less, 50 weight percent or less, 40 weight percent or less, or 30 weight percent or less) relative to the HNO-releasing polymer.

The 1,2-oxazine moieties and the hydrophilic pendant moieties can each independently covalently bonded to the multivalent scaffold via a first linker L₁ selected from C₁₋₂₀ alkylene, C₂₋₂₀ alkenylene, C₂₋₂₀ alkynylene, an ether linkage, a carbamate linkage, a carbonate linkage, an amide linkage and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene. In some embodiments, the first linker L₁ is selected from C₁₋₂₀ alkylene, C₂₋₂₀ alkenylene, C₂₋₂₀ alkynylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, C₂₋₂₀ alkynylene, or a divalent poly(C₁₋₄ alkylene glycol) linkage. In some embodiments, the first linker L₁ is selected from C₁₋₂₀ alkylene, C₂₋₂₀ alkenylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or a divalent poly(C₁₋₄ alkylene glycol) linkage. In some embodiments, the first linker L₁ is selected from C₁₋₂₀ alkylene, a divalent poly(C₁₋₄ alkylene glycol) linkage, an amide linkage, and an ester linkage, wherein said amide linkage, or ester linkage is optionally substituted with 1 or 2 C₁₋₂₀ alkylene or a divalent poly(C₁₋₄ alkylene glycol) linkage. In some embodiments, the first linker L₁ is selected from C₁₋₂₀ alkylene, an amide linkage, and an ester linkage, wherein said amide linkage, or ester linkage is optionally substituted with 1 or 2 C₁₋₂₀ alkylene. In some embodiments, the first linker L₁ is a C₁₋₂₀ alkylene.

In some embodiments, the HNO-releasing compound further includes a second linker L₂ between the 1,2-oxazine moiety and the first linker L₁. The second linker L₂ can be selected from C₁₋₂₀ alkylene, C₂₋₂₀ alkenylene, C₂₋₂₀ alkynylene, an ether linkage, a carbamate linkage, a carbonate linkage, an amide linkage and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene. In some embodiments, the second linker L₂ is selected from C₁₋₂₀ alkylene, C₂₋₂₀ alkenylene, C₂₋₂₀ alkynylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, C₂₋₂₀ alkynylene, or a divalent poly(C₁₋₄ alkylene glycol) linkage. In some embodiments, the second linker L₂ is selected from C₁₋₂₀ alkylene, C₂₋₂₀ alkenylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or a divalent poly(C₁₋₄ alkylene glycol) linkage. In some embodiments, the second linker L₂ is selected from C₁₋₂₀ alkylene, a divalent poly(C₁₋₄ alkylene glycol) linkage, an amide linkage, and an ester linkage, wherein said amide linkage, or ester linkage is optionally substituted with 1 or 2 C₁₋₂₀ alkylene or a divalent poly(C₁₋₄ alkylene glycol) linkage. In some embodiments, the second linker L₂ is selected from C₁₋₂₀ alkylene, an amide linkage, and an ester linkage, wherein said amide linkage, or ester linkage is optionally substituted with 1 or 2 C₁₋₂₀ alkylene. In some embodiments, the second linker L₂ is a C₁₋₂₀ alkylene.

In some embodiments, the compound of Formula (I) is a polymer including a repeat unit of Formula (III)

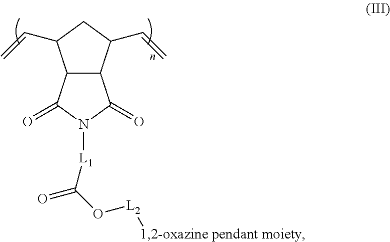

wherein the 1,2-oxazine pendant moiety has any one of Formula (I-A), (I-B), (I-C), (I-D), (I-E), and (I-F) as defined above, and

L₁ and L₂ are as defined above.

In some embodiments, the compound of Formula (I) is a polymer including a repeat unit of Formula (IV)

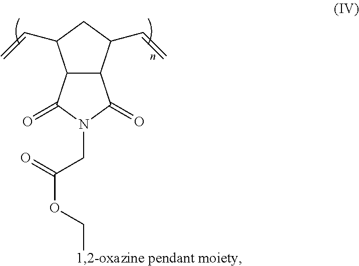

wherein the 1,2-oxazine pendant moiety has any one of Formula (I-A), (I-B), (I-C), (I-D), (I-E), or (I-F), as defined above.

In some embodiments, the polymer of including a repeating unit of Formula (III) and/or (IV) further includes a repeat unit of Formula (V)

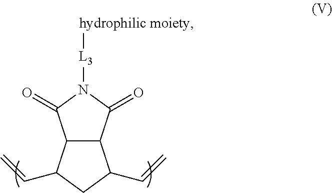

wherein L₃ is selected from C₁₋₂₀ alkylene, C₂₋₂₀ alkenylene, C₂₋₂₀ alkynylene, an ether linkage, a carbamate linkage, a carbonate linkage, an amide linkage and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene. In some embodiments, L₃ is selected from C₁₋₂₀ alkylene, C₂₋₂₀ alkenylene, C₂₋₂₀ alkynylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, C₂₋₂₀ alkynylene, or a divalent poly(C₁₋₄ alkylene glycol) linkage. In some embodiments, L₃ is selected from C₁₋₂₀ alkylene, C₂₋₂₀ alkenylene, a divalent poly(C₁₋₄ alkylene glycol) linkage, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or a divalent poly(C₁₋₄ alkylene glycol) linkage. In some embodiments, L₃ is selected from C₁₋₂₀ alkylene, a divalent poly(C₁₋₄ alkylene glycol) linkage, an amide linkage, and an ester linkage, wherein said amide linkage, or ester linkage is optionally substituted with 1 or 2 C₁₋₂₀ alkylene or a divalent poly(C₁₋₄ alkylene glycol) linkage. In some embodiments, L₃ is selected from C₁₋₂₀ alkylene, an amide linkage, and an ester linkage, wherein said amide linkage, or ester linkage is optionally substituted with 1 or 2 C₁₋₂₀ alkylene. In some embodiments, L₃ is a C₁₋₂₀ alkylene.

In certain embodiments, the polymers of the present disclosure are according to Formula (VI):

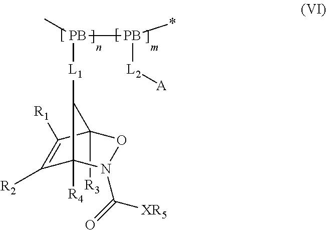

wherein

PB is the polymer backbone;

n is a number selected from 5-10,000;

L₁, L₂, R₁, R₂, R₃, R₄, and R₅ are each as defined above;

X is selected from NH and O; and

A is a hydrophilic group.

In certain embodiments, the mole ratio of n:m is configured to render the polymer of Formula (VI) soluble in an aqueous or biological liquid. Accordingly, in certain embodiments, the mole ratio of n:m is approximately 0.5:1, 0.75:1, 0.85:1, 0.9:1, 0.95:1, 0.99:1, 1:1, 1:0.99, 1:0.95, 1:0.9, 1:0.85, 1:0.75, and 1:0.5. The polymer of Formula (VI) can be a block copolymer (having n and m blocks), or a random copolymer, where the repeating units having n and m subscripts are randomly distributed in the polymer backbone.

In certain embodiments, the co-polymer is according to Formula (VII):

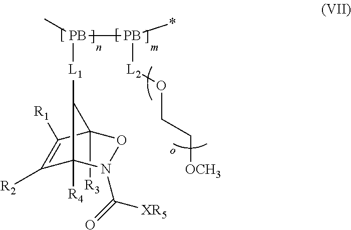

wherein

PB is the polymer backbone;

n is a number selected from 5-10,000;

L₁, L₂, R₁, R₂, R₃, R₄, and R₅ are each as defined above;

X is selected from NH and O; and

A is a hydrophilic group.

m is a number between 5-10,000 and

o is a number between 4-30.

In certain embodiments, the mole ratio of n:m is configured to render the polymer of Formula (VII) soluble in an aqueous or biological liquid. Accordingly, in certain embodiments, the mole ratio of n:m is approximately 0.5:1, 0.75:1, 0.85:1, 0.9:1, 0.95:1, 0.99:1, 1:1, 1:0.99, 1:0.95, 1:0.9, 1:0.85, 1:0.75, and 1:0.5. The polymer of Formula (VII) can be a block copolymer (having n and m blocks), or a random copolymer, where the repeating units having n and m subscripts are randomly distributed in the polymer backbone.

In certain embodiments, the polymers of the present disclosure are according to Formula (VIII):

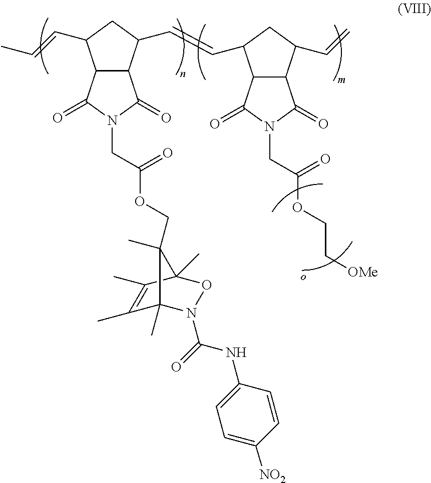

wherein

n is a number between 5-10,000;

m is a number between 5-10,000; and

o is a number between 4-30.

In certain embodiments, the mole ratio of n:m is configured to render the polymer of Formula (VIII) soluble in an aqueous or biological liquid. Accordingly, in certain embodiments, the mole ratio of n:m is approximately 0.5:1, 0.75:1, 0.85:1, 0.9:1, 0.95:1, 0.99:1, 1:1, 1:0.99, 1:0.95, 1:0.9, 1:0.85, 1:0.75, and 1:0.5. The polymer of Formula (VIII) can be a block copolymer (having n and m blocks), or a random copolymer, where the repeating units having n and m subscripts are randomly distributed in the polymer backbone.

In some embodiments, the polymer of the present disclosure is a block-copolymer. In some embodiments, the polymer of the present disclosure is a random copolymer.

In some embodiments, the decomposition of the 1,2-oxazine moiety is triggered by a stimulus such as heat, UV-irradiation, or both.

### Signaling and Therapeutic Agents

In certain embodiments, the signaling agent is selected from enzymes, prosthetic groups, fluorescent materials, luminescent materials, and bioluminescent materials.

In certain embodiments, the therapeutic agent is selected from a small molecule, a polymer, a peptide, a nucleic acid, a lipid, and a carbohydrate.

Protein Agents.

In some embodiments, the therapeutic agent is a protein or peptide. The terms “protein,” “polypeptide,” and “peptide” can be used interchangeably. In certain embodiments, peptides range from about 5 to about 5000, 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size.

Polypeptides can contain L-amino acids, D-amino acids, or both and can contain any of a variety of amino acid modifications or analogs known in the art. Useful modifications include, e.g., terminal acetylation, amidation. In some embodiments, polypeptides can include natural amino acids, unnatural amino acids, synthetic amino acids, and combinations thereof, as described herein.

In some embodiments, the therapeutic agent is a hormone, erythropoietin, insulin, cytokine, antigen for vaccination, and/or growth factor. In some embodiments, the therapeutic agent can be an antibody and/or characteristic portion thereof. In some embodiments, antibodies include, but are not limited to, polyclonal, monoclonal, chimeric (i.e., “humanized”), or single chain (recombinant) antibodies. In some embodiments, antibodies have reduced effector functions and/or bispecific molecules. In some embodiments, antibodies include Fab fragments and/or fragments produced by a Fab expression library (e.g. Fab, Fab′, F(ab′)2, scFv, Fv, dsFv diabody, and Fd fragments).

Nucleic Acid Agents.

In certain embodiments, the therapeutic agent is a nucleic acid (e.g., DNA, RNA, derivatives thereof). In some embodiments, the nucleic acid agent is a functional RNA. In general, a “functional RNA” is an RNA that does not code for a protein but instead belongs to a class of RNA molecules whose members characteristically possess one or more different functions or activities within a cell. It will be appreciated that the relative activities of functional RNA molecules having different sequences can differ and can depend at least in part on the particular cell type in which the RNA is present. Thus the term “functional RNA” is used herein to refer to a class of RNA molecule and is not intended to imply that all members of the class will in fact display the activity characteristic of that class under any particular set of conditions. In some embodiments, functional RNAs include RNAi-inducing entities (e.g., short interfering RNAs (siRNAs), short hairpin RNAs (shRNAs), and microRNAs), ribozymes, tRNAs, rRNAs, RNAs useful for triple helix formation.

Carbohydrate Agents.

In some embodiments, the therapeutic agent is a carbohydrate. In certain embodiments, the carbohydrate is a carbohydrate that is associated with a protein (e.g. glycoprotein, proteogycan). A carbohydrate can be natural or synthetic. A carbohydrate can also be a derivatized natural carbohydrate. In certain embodiments, a carbohydrate can be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, malitol, and lactitol.

Lipid Agents.

In some embodiments, the therapeutic agent is a lipid. In certain embodiments, the lipid is a lipid that is associated with a protein (e.g., lipoprotein). Exemplary lipids that can be used in accordance with the present disclosure include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g., vitamin E), phospholipids, sphingolipids, and lipoproteins.

In some embodiments, the lipid can include one or more fatty acid groups or salts thereof. In some embodiments, the fatty acid group include digestible, long chain (e.g., C8-C50), substituted or unsubstituted hydrocarbons. In some embodiments, the fatty acid group is one or more of butyric, caproic, caprylic, capric, lauric, myristic, palmitic, stearic, arachidic, behenic, or lignoceric acid. In some embodiments, the fatty acid group is one or more of palmitoleic, oleic, vaccenic, linoleic, alpha-linolenic, gamma-linoleic, arachidonic, gadoleic, arachidonic, eicosapentaenoic, docosahexaenoic, or erucic acid.

Small Molecule Agents.

In some embodiments, the therapeutic agent is a small molecule and/or organic compound with pharmaceutical activity. In some embodiments, the therapeutic agent is a clinically-used drug. In some embodiments, the drug is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g., cell growth inhibitors and anti-adhesion molecules), inhibitor of DNA, RNA, or protein synthesis.

In certain embodiments, the small molecule therapeutic agent is an NSAID such as a salicylate; a proprionic acid derivative such as ibuprofen or (S)-naproxen; an acetic acid derivative such as indomethacin; anthranilic acid derivatives such as meclofenamic acid; COX-2 inhibitors such as celecoxib; and other secondary sulfonamides such as sumatriptan, topiramate, and acetazolamide.

In certain embodiments, a small molecule agent is any drug. In some embodiments, the drug is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present disclosure.

A more complete listing of classes and specific drugs suitable for use in the present disclosure may be found in Pharmaceutical Drugs: Syntheses, Patents, Applications by Axel Kleemann and Jurgen Engel, Thieme Medical Publishing, 1999; and the Merck Index: An Encyclopedia of Chemicals, Drugs and Biologicals, Ed. by Budavari et al., CRC Press, 1996, both of which are incorporated herein by reference.

### Compositions and Kits

In yet another aspect, the present disclosure features a composition, including a triggering agent selected from a photothermal dye and a nanoparticle; and the HNO-releasing compound described above. As used herein, a photothermal dye refers to a small molecule configured to convert photonic energy to thermal energy. In some embodiments, the photothermal dye is selected from IR-780, IR-808, indocyanine green (ICG), and croconaine dyes. In some embodiments, the nanoparticle is a gold nanoparticle. The nanoparticles and/or the photothermal dyes can be coupled to a targeting agent, such as an antibody, useful in selectively binding the nanoparticles and/or the photothermal dyes to a target cell population or tissue.

In a further aspect, the present disclosure features kits including the HNO-releasing compounds as described above and the nanoparticles and/or photothermal dyes configured to convert photonic energy to thermal energy. Such kits are useful in, for example, photothermal therapy. The contents of the kits can be administered locally or generally to a subject and photonic energy is delivered to one or more sites of interest in the subject for treatment. When sufficient photonic energy is delivered to the one or more sites the nanoparticles and/or the photothermal dyes absorb light and convert at least a portion of the absorbed light into heat. The 1,2-oxazine moieties of the present disclosure then degrade as a result of the heat to produce HNO and, in certain embodiments, a therapeutic and/or signaling agent. In certain embodiments, the therapeutic agent is an anti-cancer or anti-tumor agent.

### Use

In use, the compounds of the present disclosure can be exposed to a triggering event (i.e., a stimulus), which decomposes the compounds as shown, for example, in FIGS. 1 and 2, and releases HNO from the compounds. Carbon dioxide can also be released from the compounds of the present disclosure. In some embodiments, therapeutic agents, signaling agents, and/or fluorescent molecules are released from the compounds. The release of the therapeutic agents, signaling agents, and/or fluorescent molecules can occur concurrently or subsequently to the release of HNO and carbon dioxide.

The compounds of the present disclosure can be used to treat a subject, by administering to the subject a therapeutically effective amount of one or more of the compounds of the present disclosure or one of the kits of the present disclosure, to release HNO, CO₂, therapeutic agents, signaling agents, and/or fluorescent molecules to treat the subject. During treatment, heat can be administered to a subject or to a portion of the subject to decompose one or more of the 1,2-oxazine moieties on the multivalent scaffold. In some embodiments, light can be administered to a subject or to a portion of the subject to heat a nanoparticle and/or a photothermal dye that is also administered to a subject, and thereby trigger decomposition of the 1,2-oxazine moieties on the multivalent scaffold.

The synthesis and characterization of polymers including pendant 1,2-oxazine moieties are provided in the Example. The 1,2-oxazine moieties decompose upon exposure to thermal energy.

### Example

Oxazines can serve thermal triggers for self-immolative polymers (SIPs), where the oxazine was derived from a peralkylated cyclopentadiene and a carbamoylnitroso species generated in situ from the corresponding hydroxy urea. When used as a thermal trigger for SIPs, a single oxazine unit is capable of triggering depolymerization of an entire polymer block. The small molecule constituents that are released, however, would require significant modification to enable therapeutic relevance, and the potential synergistic or parallel benefits of HNO release are precluded by the low oxazine content (one per polymer chain). It is believed that HNO-releasing multivalent scaffolds such as polymer scaffolds can provide significant therapeutic benefits. Herein, the potential of 1,2-oxazine adducts as a thermally sensitive moiety capable of releasing covalently attached small molecules is demonstrated.

In the polymers of present example, thermolysis of the covalently-bound oxazine adduct liberates a drug mimic molecule as well as an equivalent of nitroxyl (HNO) which is in of itself a proposed therapeutic for cancer and various cardiovascular-related issues. It is believed that multiple equivalents of HNO release from a polymer-based drug delivery system is as of yet unrealized. Furthermore, by using 1,2-oxazines as the covalently labile moiety, HNO effectively serves as a traceless and benign linker between carrier (polymer) and cargo (therapeutic small molecule). This significantly limits unwanted and potentially detrimental side reactions of reactive by-products and intermediates of the covalently labile linker.

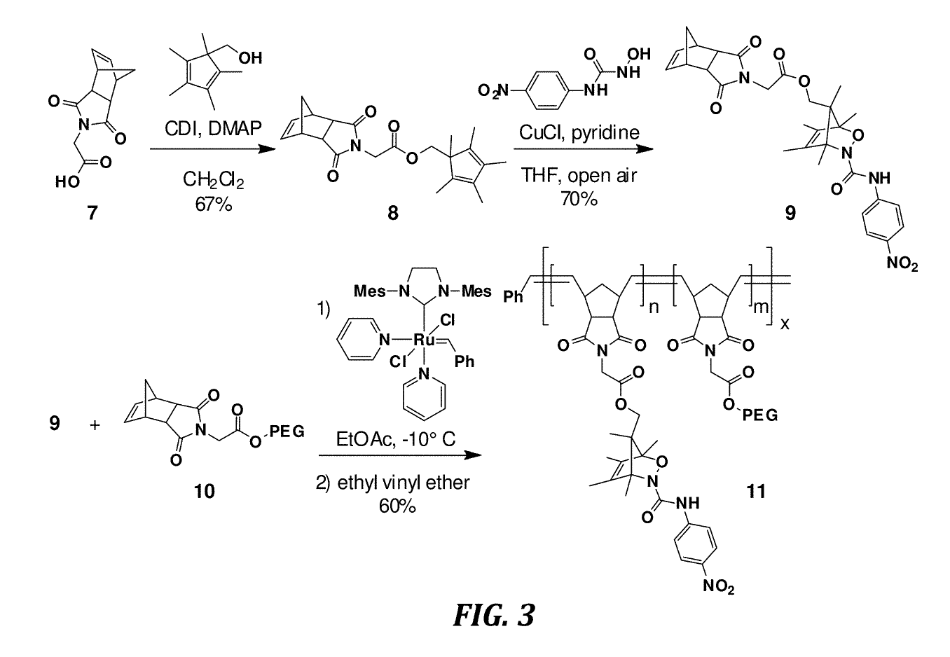

Ring opening metathesis polymerization (ROMP) was found to be a successful strategy for norbornene-based monomers bearing oxazine moieties (FIG. 3). Starting from known norbornene 7, 1′-Carbonyldiimidazole (“CDI”) coupling furnished the cyclopentadiene-functionalized norbornene 8. Subsequent reaction with N-(p-nitrophenyl)hydroxy urea in the presence of Cu(I) salts provided the desired monomer (9) with oxazine adducts in 49% overall yield from 7. Using a copolymerization with PEG-functionalized monomer 10 (1:3 molar ratio of 9 to 10) provided a water soluble copolymer (11). Polymer 11 was analyzed by GPC and found to have Mₙ=34.6 kDa and D=1.04. By ¹H NMR analysis, an average of 16 oxazine units was calculated to be present per polymer chain.

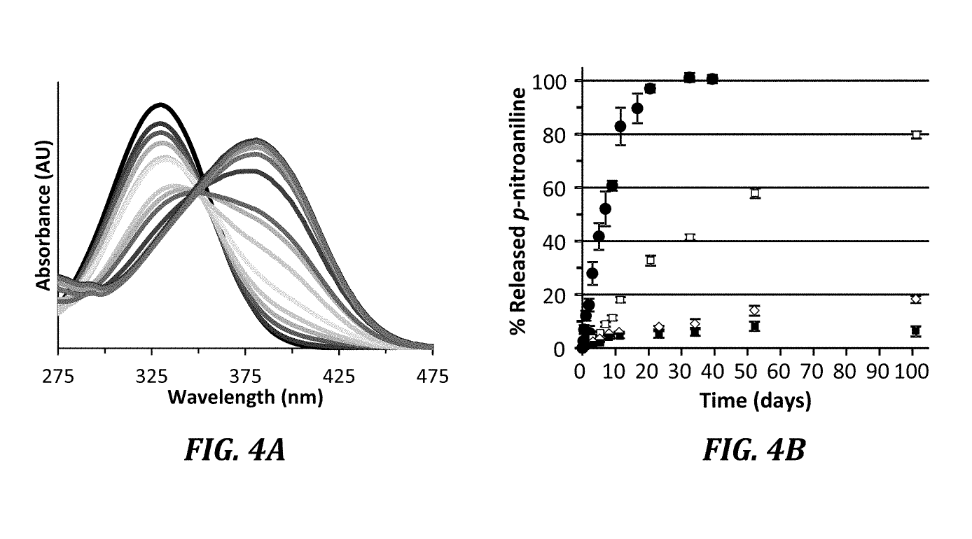

The polymers were then dissolved in phosphate buffered H₂O and the release of p-nitroaniline was monitored by UV-vis spectrometry at various temperatures (FIG. 4A). The oxazine adduct showed a λₘₐₓ near 329 nm, whereas the released p-nitroaniline showed a λₘₐₓ near 390 nm. A representative set of UV-vis spectra are shown in FIG. 4A (left), and revealed a steady decrease in the amount of oxazine adduct with concomitant increase in the absorbance that corresponds to p-nitroanline. Confirmation of p-nitroaniline production was further corroborated by ¹H NMR spectroscopy. In FIG. 4B is shown the % of released p-nitroaniline over time at various temperatures. All samples were evaluated in pH 7.5 buffered water. At a cold storage temperature of 4° C., an expectedly slow release (black solid squares) was observed. Similarly, at 22° C. less than 20% production of p-nitroaniline after 100 days was observed. Increasing the temperature to 37° C. increased the rate of release considerably, and a steady kinetic profile was observed over the experiment time course of 100 days. This is exciting in that it signifies an ability for sustained release of HNO and small molecule organics (or SIP activation) at physiological temperatures. As expected, increasing the temperature to 60° C. resulted in considerably faster breakdown of the adduct, which indicated that local heating techniques or photothermal transduction may be a viable method for augmenting the oxazine activation in these systems.

Thus, water soluble, oxazine-containing poly(norbornene) polymers were demonstrated as successful platforms for the controlled and sustained release of small molecules. These polymers can undergo a sustained release of covalently attached small molecules at physiological temperatures over the course of several days. The temperature-dependent release profiles potentiate external control via application of hyperthermic techniques. Additionally, evidence for concomitant HNO production provides therapeutic potential to these polymer systems.

### Experimental Details

General Considerations.

Dry tetrahydrofuran (THF), pyridine, and CH₂Cl₂ were obtained from a Glass Contour solvent purification system. All other reagents and solvents were used as obtained from commercial sources. ¹H and ¹³C NMR spectra were recorded on a Bruker AVance 300 or 500 MHz spectrometer. Chemical shifts are reported in delta (δ) units, expressed in parts per million (ppm) downfield from tetramethylsilane using the residual protio-solvent as an internal standard (CDCl₃, ¹H: 7.26 ppm and ¹³C: 77.16 ppm). LRMS was performed on a Bruker Esquire equipped with either an electrospray ionization (ESI) or IonSense SVP100 DART source. GPC setup consists of: a Shimadzu pump, three in-line MZ-Gel 10 μm size-exclusion columns (10³, 10³, and 10⁵ Å), DAWN-HELEOS II 18-angle multi-angle laser light scatter and OptiLab T-rEx refractive index detectors (each from Wyatt Technologies Corporation). The mobile phase consisted of THF. No calibration standards were used, and do/dc values were obtained for each injection assuming 100% mass elution from the columns. UV-Vis experiments were conducted on an Agilent 8453 Diode Array UV-Vis Spectrophotometer.

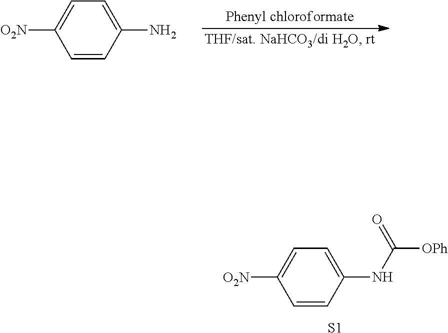

Synthesis of S1.

A round bottom flask was charged with 4-nitroaniline (6.0 g, 43.4 mmol, 1.0 eq.), stir bar, 36 mL of THF, 36 mL of sat. NaHCO₃ and 18 mL of diH₂O. Phenyl chloroformate (5.5 mL, 44.3 mmol, 1.0 eq.) was added drop-wise via an addition funnel. The reaction mixture was allowed to stir at room temperature for 3 hours. To the reaction mixture, ethyl acetate (EtOAc) was added and the organic layer was washed with sat. NaHCO₃ (2×20 mL) and brine (1×20 mL). The organic layer was dried over Na₂SO₄ and concentrated under reduced pressure. The resulting carbamate was used without further purification (93% yield). ¹H NMR (500 MHz, CDCl₃) δ 8.21 (d, J=9.0 Hz, 2H), 7.60 (d, J=9.0 Hz, 2H), 7.49-7.35 (m, 3H), 7.30-7.24 (m, 1H), 7.19 (d, J=7.8 Hz, 2H). ¹³C NMR (126 MHz, CDCl₃) δ 151.30, 150.24, 143.55, 143.50, 129.74, 126.39, 125.39, 121.59, 118.22.

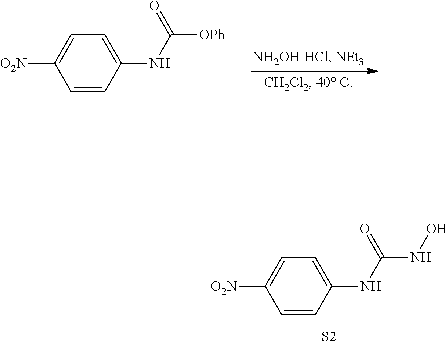

Synthesis of S2.

Into a flame-dried, N₂-purged round bottom flaks was added carbamate 51 (2.0 g, 6.9 mmol, 1.0 eq.), NEt₃ (1.0 mL, 6.9 mmol, 1.0 eq.), CH₂Cl₂ (7 mL) and stir bar. Hydroxylamine (687.0 mg, 20.8 mmol, 3.0 eq.) in a solution of dry dimethyl sulfoxide (DMSO) (10.4 mL) was then added. The reaction mixture was heated to 40° C. and stirred for 3 hours. The reaction mixture was then added into 200 mL of diH₂O and extracted with EtOAc (4×50 mL). The desired product was obtained as a yellow solid (83% yield). ¹HNMR (500 MHz, DMSO) δ 9.49 (s, 1H), 9.27 (s, 1H), 9.15 (s, 1H), 8.14 (d, J=9.0 Hz, 2H), 7.92 (d, J=8.9 Hz, 2H). ¹³C NMR (126 MHz, DMSO) δ 157.82, 146.39, 141.37, 124.79, 118.47.

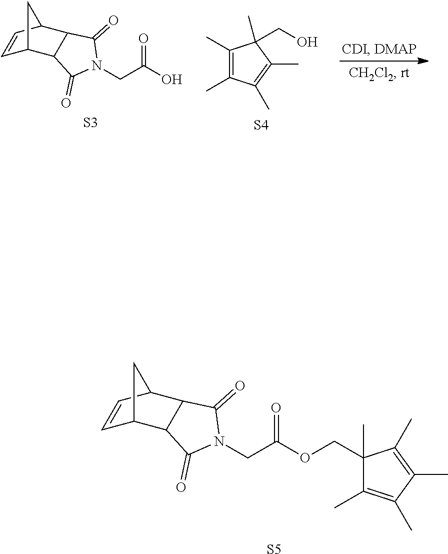

Synthesis of S5.

Compounds S3 and S4 were synthesized according to the literature. Into a flame-dried, N₂-purged round bottom flask, N,N-carbonyldiimidazole (CDI) (1.4 g, 8.7 mmol, 1.3 eq.), stir bar and 15 mL of CH₂Cl₂ were added. To this solution, S3 (1.9 g, 8.7 mmol, 1.3 eq.) was added portion-wise. The reaction mixture was allowed to stir at room temperature for 45 minutes. After this time, 4-dimethylaminopyridine (DMAP) (81.8 mg, 0.7 mmol, 0.1 eq.) and S4 (1.1 g, 6.7 mmol, 1.0 eq.) in 15 mL of CH₂Cl₂ were added to the reaction mixture. The reaction mixture was allowed to stir at room temperature for 18 hours. The reaction mixture was then washed with 1 M HCl (2×30 mL), sat. NaHCO₃ (2×30 mL) and brine (2×30 mL). The organic layer was dried over Na₂SO₄ and concentrated under reduced pressure. The resulting residue was purified by flash chromatography (10% EtOAc/Hex) and the product was isolated as a white solid (70% yield). ¹H NMR (300 MHz, CDCl₃) δ 6.30 (s, 2H), 4.14 (s, 2H), 4.00 (s, 2H), 3.30 (s, 2H), 2.72 (s, 2H), 1.74 (d, J=10.5 Hz, 12H), 1.65 (d, J=10.0 Hz, 1H), 1.52 (d, J=12.5 Hz, 1H), 0.94 (s, 3H). ¹³C NMR (126 MHz, CDCl₃) δ 177.11, 166.84, 138.25, 138.06, 135.37, 69.14, 55.37, 48.10, 45.48, 42.99, 39.37, 16.97, 11.17, 10.27.

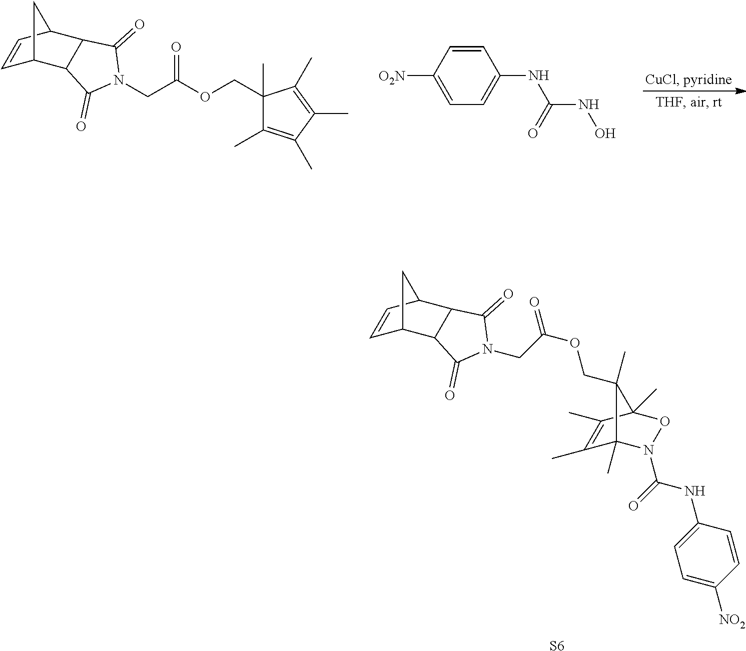 [SMILES: CC1=C(C)C(C)(COC(=O)CN2C(=O)C3C4C=CC(C4)C3C2=O)C(C)=C1C ; O=C(NO)Nc1ccc([N+](=O)[O-])cc1 ; CC1=C(C)C2(C)N(C(=O)Nc3ccc([N+](=O)[O-])cc3)OC1(C)C2(C)COC(=O)CN1C(=O)C2C3C=CC(C3)C2C1=O ; "S6"]

Synthesis of S6.

A round bottom flask was charged with S5 (1.5 g, 4.1 mmol, 1.0 eq.), S2 (828 mg, 4.9 mmol, 1.2 eq.) stir bar and THF (20 mL). CuCl (96 mg, 0.97 mmol, 0.24 eq.) and pyridine (19 μL, 0.24 mmol, 0.06 eq.) were then added. The reaction mixture was vigorously stirred under ambient conditions for 18 hours. Ethyl acetate (20 mL) and sat. EDTA (20 mL) were then added to the reaction mixture. The organic layer was washed with sat. EDTA (2×20 mL) and brine (2×20 mL). The organic layer was dried over Na₂SO₄ and then concentrated under reduced pressure. The crude material was purified by flash chromatography (20% EtOAc/Hex) and the product was isolated as a yellow solid (70% yield). ¹H NMR (500 MHz, CDCl₃) δ 8.10 (d, 2H), 7.80 (s, 1H), 7.53 (d, 2H), 6.27 (s, 2H), 4.20-4.14 (m, 2H), 3.84 (m, 2H), 3.27 (s, 2H), 2.73 (s, 2H), 1.80-1.63 (m, 10H), 1.49 (m, 1H), 1.41 (m, 1H), 1.38 (s, 2H), 1.05 (s, 2H), 0.73 (s, 1H). ¹³C NMR (126 MHz, CDCl₃) δ 177.06, 166.85, 159.76, 143.80, 143.01, 139.09, 137.98, 132.56, 125.02, 118.54, 96.86, 96.29, 81.32, 68.00, 66.43, 62.06, 60.77, 48.06, 45.44, 42.84, 39.24, 12.97, 12.70, 12.59, 12.49, 12.01, 11.02, 10.84, 9.31.

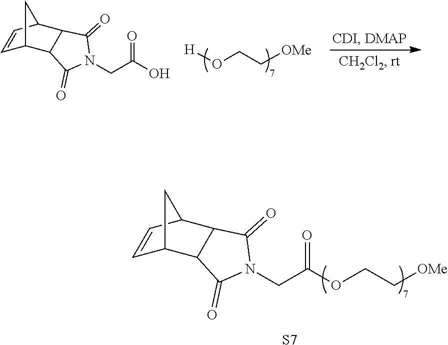

Synthesis of S7.

Into a flame-dried, N₂-purged round bottom flask, N,N-carbonyldiimidazole (1.7 g, 10.7 mmol, 1.3 eq.), stir bar and 20 mL of CH₂Cl₂ were added. To this solution, S7 (2.4 g, 10.7 mmol, 1.3 eq.) was added portion-wise. The reaction mixture was allowed to stir at room temperature for 45 minutes. After this time, DMAP (100 mg, 0.82 mmol, 0.1 eq.) and poly(ethylene glycol) methyl ether (350 Da) (3.0 mL, 8.2 mmol, 1.0 eq.) in 20 mL of CH₂Cl₂ were added to the reaction mixture. The reaction mixture was allowed to stir at room temperature for 18 hours. The reaction mixture was then concentrated under reduced pressure and pushed through an alumina/celite plug, using CH₂Cl₂ (˜60 mL) as eluent. The solution was then concentrated under reduced pressure to afford pure product. (95% yield)¹H NMR (500 MHz, CDCl3) δ 6.23 (s, 1H), 4.25-4.20 (m, 1H), 4.18 (s, 1H), 3.57 (d, J=4.8 Hz, 15H), 3.30 (s, 1H), 3.24 (s, 1H), 2.68 (s, 1H), 1.65 (d, J=10.0 Hz, 1H), 1.45 (d, J=10.0 Hz, 1H). ¹³C NMR (126 MHz, CDCl3) δ 176.53, 166.54, 137.58, 71.49, 70.13, 68.31, 64.45, 58.54, 47.54, 44.98, 42.44, 38.95.

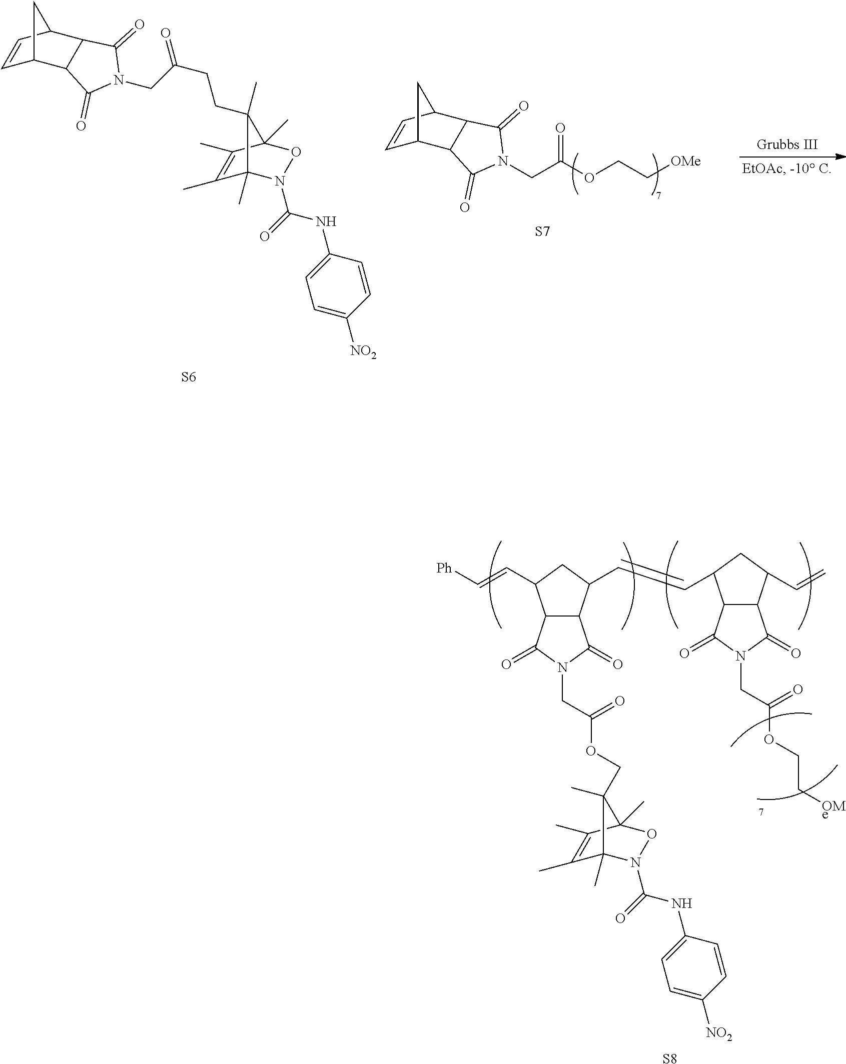

Synthesis of S8:

Into a flame-dried, N₂-purged round bottom flask, monomers S6 (75 mg, 0.13 mmol, 15 eq.), S7 (216 mg, 0.39 mmol, 45 eq.), stir bar and 15 mL of N₂-purged EtOAc were added. Once dissolved, the solution was cooled to −10° C. and Grubbs 3^{rd} generation catalyst (dichloro[1,3-bis(2,4,6-trimethylphenyl)-2-imidazolidinylidene](benzylidene)bis(3-bromopyridine)ruthenium(II)) (6.3 mg, 0.0087 mmol, 1.0 eq.) in 0.87 mL of N₂-purged EtOAc was quickly added. The reaction solution was allowed to stir for 2 hours, letting the ice bath expire. Ethyl vinyl ether (2.4 mL) was added to the reaction and the solution was stirred for an additional 10 minutes. The reaction mixture was then concentrated under reduced pressure and precipitated into diethyl ether. The ether solution was decanted away from the polymer residue. The polymer was then dissolved in minimal CH₂Cl₂ and pushed through an alumina/Celite plug, using CH₂Cl₂ as eluent. The solution was again concentrated under reduced pressure and precipitated into diethyl ether, providing gooey polymer (173 mg, 60% yield). Mn=34.6 kDa, D=1.02. A 1:3 ratio of S6 to S7 in the polymer was confirmed by ¹H NMR spectroscopy. Average oxazine content per polymer chain is ˜16.4.

General Method for Monitoring the Release of 4-Nitroaniline from S8

Into a 100 mL volumetric flask was added 2.7 mL of S8 (1.12×10⁻⁴ M) and 0.5 mL of 100 mM pH 7.5 phosphate buffer which was then diluted with 100 mL of Milli-Q H₂O. The resulting solution was then filtered through a 0.45 μm pore-size poly(tetrafluoroethylene) PTFE syringe filter. The polymer solution was then portioned out (5 mL) into screw-cap scintillation vials and sealed with Teflon-lined lids. The vials were then submerged in a pre-heated oil bath where applicable. Samples were analyzed by UV-vis spectrometry.

Except as otherwise noted, the methods and techniques of the present embodiments are generally performed according to conventional methods well known in the art and as described in various general and more specific references that are cited and discussed throughout the present specification.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A HNO-releasing compound, comprising
(a) a multivalent scaffold; and
(b) a plurality of 1,2-oxazine pendant moieties independently covalently bound to the scaffold, the 1,2-oxazine pendant moieties having any one of Formula (I-A), (I-B), (I-C), (I-D), (I-E), or (I-F), 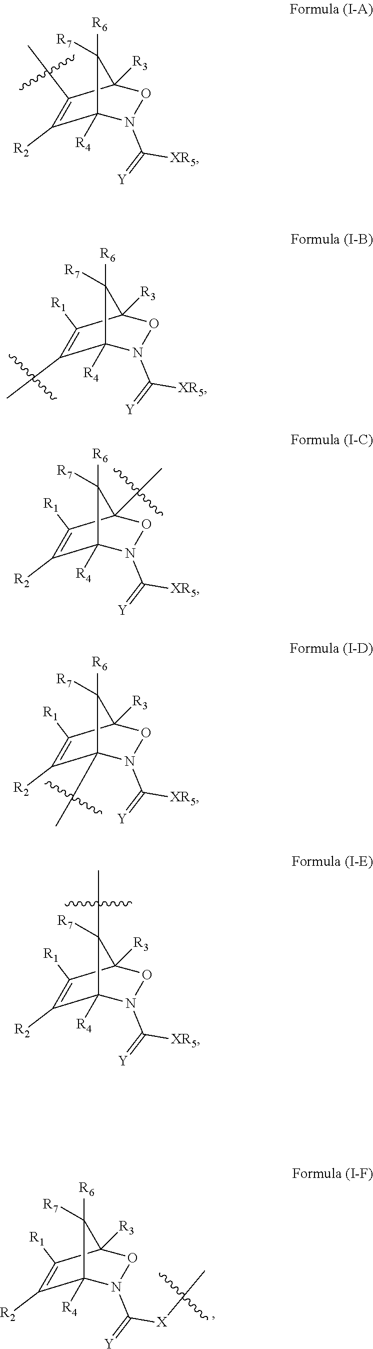
wherein
R₁, when present, is independently selected from a fluorescent molecule, a signaling agent, and a therapeutic agent, each optionally covalently bonded to a self-immolative linker, or R₁ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
R₂, when present, is independently selected from a fluorescent molecule, a signaling agent, and a therapeutic agent, each optionally covalently bonded to a self-immolative linker, or R₂ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
R₃, when present, is independently selected from a fluorescent molecule, a signaling agent, and a therapeutic agent, each optionally covalently bonded to a self-immolative linker, or R₃ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
R₄, when present, is independently selected from a fluorescent molecule, a signaling agent, and a therapeutic agent, each optionally covalently bonded to a self-immolative linker, or R₄ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
R₆, when present, is independently selected from a fluorescent molecule, a signaling agent, and a therapeutic agent, each optionally covalently bonded to a self-immolative linker, or R₆ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
R₇, when present, is independently selected from a fluorescent molecule, a signaling agent, and a therapeutic agent, each optionally covalently bonded to a self-immolative linker, or R₇ is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
R₅, when present, is selected from a fluorescent molecule, a signaling agent, and a therapeutic agent, each optionally covalently bonded to a self-immolative linker; or
R₅, when present, is independently selected from H, C₁₋₆ alkyl, C₂₋₆ alkenyl, halo, aryl, and heteroaryl, wherein the C₁₋₆ alkyl, C₂₋₆ alkenyl, aryl, or heteroaryl is each optionally substituted with 1, 2, 3, or 4 substituents each independently selected from halo, hydroxy, OMe, OAc, CN, NO₂, and CO₂C₁₋₆ alkyl;
Y is selected from O, NH, NC₁₋₆alkyl, and S; and
X is selected from O and NH;
wherein each 1,2-oxazine pendant moiety is configured to release one HNO molecule upon decomposition.

2. The HNO-releasing compound of claim 1, wherein the multivalent scaffold is selected from a polymer, a nanoparticle, a micelle, a liposome, a microbubble, a hydrogel, and electrospun fiber.

3. The HNO-releasing compound of claim 1, wherein the multivalent scaffold is a polymer.

4. The HNO-releasing compound of claim 3, wherein the polymer is selected from a poly(olefin), a poly(cyclic olefin), a poly(ester), a poly(ether), a poly(urethane), a poly(acrylate), a poly(acrylamide), and a poly(C₁₋₃ alkyl acrylate).

5. The HNO-releasing compound of claim 1, wherein the polymer is a poly(cyclic olefin).

6. The HNO-releasing compound of claim 1, wherein the poly(cyclic olefin) comprises a poly(norbornene) of Formula (II) 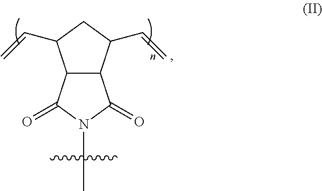
wherein n is an integer from 5-20,000.

7. The HNO-releasing compound of claim 1, wherein the 1,2-oxazine moiety is covalently bonded to the multivalent scaffold via a first linker L₁.

8. The HNO-releasing compound of claim 1, wherein the first linker L₁ is selected from C₁₋₂₀ alkylene, C₂₋₂₀ alkenylene, C₂₋₂₀ alkynylene, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene.

9. The HNO-releasing compound of claim 1, wherein the multivalent scaffold further comprises hydrophilic pendant moieties.

10. The HNO-releasing compound of claim 9, wherein the hydrophilic moieties comprise poly(ethylene glycol) or a C₁₋₃ alkoxy poly(ethylene glycol).

11. The HNO-releasing compound of claim 9, wherein the 1,2-oxazine moieties and the hydrophilic pendant moieties are each independently covalently bonded to the multivalent scaffold via a first linker L₁ independently selected from C₁₋₂₀ alkylene, C₂₋₂₀ alkenylene, C₂₋₂₀ alkynylene, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene.

12. The HNO-releasing compound of claim 11, further comprising a second linker L₂ between the 1,2-oxazine moiety and the first linker L₁.

13. The HNO-releasing compound of claim 12, wherein the second linker L₂ is selected from C₁₋₂₀ alkylene, C₂₋₂₀ alkenylene, C₂₋₂₀ alkynylene, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene.

14. The HNO-releasing compound of claim 1, wherein at least one of R₁, R₂, R₃, R₄, R₅, R₆, or R₇, when present, is selected from a fluorescent molecule, a signaling agent, and a therapeutic agent, each optionally covalently bonded to a self-immolative linker, and the 1,2-oxazine moiety is configured to release the fluorescent molecule, the signaling agent, or the therapeutic agent.

15. The HNO-releasing compound of claim 14, wherein the therapeutic agent is selected from an analgesic agent, an anticancer therapeutic agent, and a non-steroidal anti-inflammatory drug (NSAID).

16. The HNO-releasing compound of claim 1, wherein the therapeutic agent is selected from salicylate NSAID, propionic acid NSAID, acetic acid NSAID derivatives, anthranilic acid NSAID.

17. The HNO-releasing compound of claim 1, wherein the therapeutic agent is selected from p-nitroaniline, ibuprofen, (S)-naproxen, indomethacin, meclofenamic acid, doxorubicin and celecoxib.

18. The HNO-releasing compound of claim 13, wherein the compound is a polymer comprising a repeat unit of Formula (III)

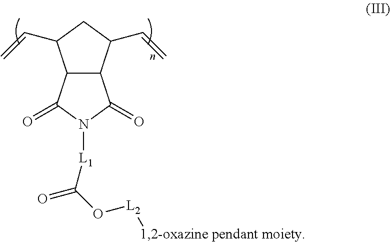

19. The HNO-releasing compound of claim 1, wherein the compound is a polymer comprising a repeat unit of Formula (IV)

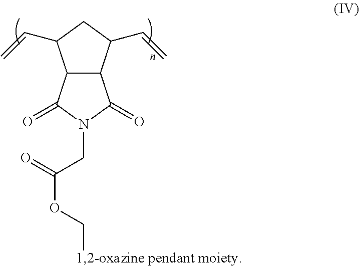

20. The HNO-releasing compound of claim 18, wherein polymer further comprises a repeat unit of Formula (V) 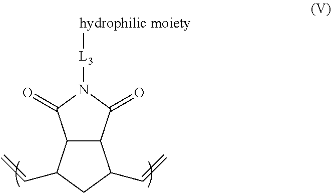
wherein L₃ is selected from C₁₋₂₀ alkylene, C₂₋₂₀ alkenylene, C₂₋₂₀ alkynylene, an ether linkage, a carbamate linkage, a carbonate linkage, an amide linkage, and an ester linkage, wherein said ether linkage, carbamate linkage, carbonate linkage, amide linkage, or ester linkage is optionally substituted with 1 or 2 C₁₋₂₀ alkylene, C₂₋₂₀ alkenylene, or C₂₋₂₀ alkynylene.

21. The HNO-releasing compound of claim 3, wherein the polymer is a block-copolymer.

22. The HNO-releasing compound of claim 3, wherein the polymer is a random copolymer.

23. The HNO-releasing compound of claim 1, wherein the decomposition is triggered by heat, UV-irradiation, or both.

24. A composition, comprising
a triggering agent selected from a photothermal dye and a nanoparticle; and
the HNO-releasing compound of claim 1.

25. The composition of claim 24, wherein the photothermal dye is selected from IR-780, IR-808, indocyanine green (ICG), and croconaine dyes.

26. The composition of claim 24, wherein the nanoparticle is a gold nanoparticle.

27. A method of releasing HNO, comprising:
exposing a compound of claim 1 to a triggering event,
decomposing a compound of claim 1, and
releasing HNO from the compound of claim 1.

28. The method of claim 27, further comprising releasing carbon dioxide from the compound of claim 1.

29. The method of claim 27, further comprising releasing a fluorescent molecule, a signaling agent, or a therapeutic agent from the compound of claim 1, wherein at least one of R₁, R₂, R₃, R₄, R₅, R₆, or R₇, when present in the compound of claim 1, is selected from a fluorescent molecule, a signaling agent, and a therapeutic agent, each optionally covalently bonded to a self-immolative linker.
